(19) United States
(12) Patent Application Publication
(10) Pub. No.: US 2020/0375447 A1
(43) Pub. Date: Dec. 3, 2020

(54) FORCE-SENSITIVE LARYNGOSCOPE SENSOR

(71) Applicant: Baylor College of Medicine, Houston, TX (US)

(72) Inventors: Kevin Rody Kotamarti, Houston, TX (US); Aaron Kotamarti, Houston, TX (US)

(73) Assignee: Baylor College of Medicine, Houston, TX (US)

(21) Appl. No.: 16/636,787

(22) PCT Filed: Aug. 3, 2018
(86) PCT No.: PCT/US18/45189

§ 371 Date: Feb. 5, 2020

Related U.S. Application Data
(60) Provisional application No. 62/542,145, filed on Aug. 7, 2017.

(51) Int. Cl. A61B 1/267; A61B 1/00; G01L 1/22

(52) U.S. Cl. CPC A61B 1/267; A61B 1/00032; G01L 1/2256; A61B 1/00055; A61B 1/00114; A61B 1/00103

(57) ABSTRACT

Force sensing may be integrated with a laryngoscope and information conveyed to the user when an applied force in a patient's mouth may cause dental trauma. By warning the user, the user may be interrupted before dental trauma occurs, allowing the user to adjust the procedure.

### CROSS-REFERENCE TO RELATED APPLICATIONS

This application is entitled to the benefit of priority to U.S. Patent Provisional Patent Application No. 62/542,156 filed on Aug. 7, 2017 entitled “Force-Sensitive Laryngoscope Sensor,” which is incorporated by reference herein.

### FIELD OF INVENTION

The present invention relates generally to therapeutic intubation devices and methods. More particularly, but not by way of limitation, the present invention relates to an apparatus for use in the intubation of patients such that the risk of dental damage is reduced.

### DESCRIPTION OF RELATED ART

Endotracheal intubation is a common medical procedure involving the placement of a flexible plastic tube (e.g., an endotracheal tube (ETT)) into the trachea of a patient. The tube maintains an open airway or serves as a conduit through which drugs may be administered. Intubation is commonly performed on critically injured or anesthetized patients to facilitate ventilation of the lungs, thereby preventing asphyxiation or airway obstruction. One conventional form of endotracheal intubation is orotracheal, in which an endotracheal tube is passed though the mouth into the trachea. Orotracheal intubation is usually performed after administration of general anesthesia and a neuromuscular-blocking drug, although it may be performed on a conscious patient via local or topical anesthesia or without any anesthesia in an emergency. Once the patient is intubated, the ETT can be connected to ventilator machines to provide artificial respiration.

During endotracheal intubation, direct laryngoscopy (DL) is employed to expose the vocal cords so that operators can view them directly to insert an ETT. One conventional technique for performing DL uses a laryngoscope. As depicted in FIG. 1, a laryngoscope includes a handle 100 and a blade 102 connected to the handle 100 at the blade attachment site 104. The blade 102 may include a horizontal flange 112, a vertical step 106, a tip 108, and a spatula 110. A battery may be located in the handle 100 to provide electricity to a light 200 located on the blade 102 by transmitting power on a conductor from the handle 100 to the blade 102 by way of a pair of electrical contacts 202, 204: one located on the distal end of the handle 204 and the other located on the proximal end of the blade 202. The blade portion 102 is attached to the handle 100 at a hinge such that the electrical contacts 202, 204 are in electrical communication with each other when the blade 102 is perpendicular to the handle 100. When the blade 102 is parallel to the handle 100, the electrical contacts 202, 204 are not in electrical communication with each other, as shown in FIG. 2.

During direct laryngoscopy (DL), a laryngoscope is passed through the oropharynx and an upwards and forward force is used to provide a clear view of the glottis. With a clear view of the glottis, the ETT can be inserted into the trachea thereby intubating the patient. In performing DL, inexperienced users may inadvertently use the maxillary central incisors as a pivot point for the laryngoscope blade, thereby causing trauma to the teeth. Such trauma may include avulsions, enamel damage, and/or implant damage. In some studies, up to or more than 10% of patients undergoing direct laryngoscopy were found to have dental trauma. Because of the frequent dental trauma, dental trauma is included as part of pre-operative consent forms patients are required to sign prior to general anesthesia.

One technique to overcome this problem is to use a mouthguard during DL. Starting in the 1950s, several mouthguards have been created with the purpose of cushioning the force from the laryngoscope blade. These have not been widely adopted largely due to the large size of the mouthguards which, when placed in an already crowded mouth, often obstruct physician's view during intubation. This reduces the physician's ability to minimize trauma. Attempts to overcome this problem have not been widely implemented, at least partially due to the attempted solutions' size, rigidity, and lack of precision.

### SUMMARY

This disclosure includes embodiments of methods, apparatuses, and systems for endotracheal intubation using a force sensitive laryngoscope sensor. A laryngoscope may include integrated force sensing. Force sensing information may be conveyed to a user of a laryngoscope when an applied force in a patient's mouth may cause dental trauma. For example, the laryngoscope may warn the user of impending dental trauma, interrupting the user before dental trauma occurs and allowing the user to adjust the procedure.

In certain embodiments, laryngoscopy devices for use in examining and performing local diagnostic and surgical procedures on the larynx may include: a body portion having an interior side and an exterior side, a force sensor coupled to the exterior side of the body portion, a circuit coupled to the force sensor, and a laryngoscope battery interface. In some embodiments, the body portion is configured to be removably connected to a laryngoscope having a laryngoscope blade such that the interior side of the body portion is adjacent to the laryngoscope blade. The force sensor may be configured to be coupled to the laryngoscope blade via a securing structure, such as a clip that couples the force sensor to the laryngoscope blade via friction. The securing structure may, for example, include an adhesive. In some embodiments, the force sensor may encase a majority or all of the laryngoscope blade.

The circuit may be configured to provide feedback based on an output of the force sensor. The circuit may include a comparator configured to detect when pressure applied to the force sensor exceeds a first threshold level and/or a second threshold level. In some embodiments, the circuit may include a microcontroller. The force sensor and the microcontroller are configured to sense when pressure is being applied to the force sensor. In some embodiments, the laryngoscope battery interface is configured to electrically connect the microcontroller to a power system of a laryngoscope when the body portion is removably connected to the laryngoscope. In some embodiments, the microcontroller may be an Atmel-based microcontroller. In other embodiments, the microcontroller may be another programmable device or logic circuitry configured to perform the functions of the microcontroller. The microcontroller may, in some embodiments, be configured to detect when pressure applied to the force sensor approaches and/or exceeds a first threshold level and a second threshold level. The microcontroller may also be configured to operate a feedback device to emit a first tone, such as a soft intermittent buzzing, as the first threshold level is reached. The microcontroller may be further configured to operate the feedback device to emit a second tone, such as a loud constant buzzing, when a second threshold level is exceeded.

The laryngoscopy device may be disposable. For example, the device may be discarded and the laryngoscope retained for further use. The body portion of the device may comprise latex rubber. The force sensor may include multiple layers of force sensitive material for sensing an applied force. In some embodiments, the force sensitive material may be conductive. The force sensor may also include non-force sensitive material, and the non-force sensitive material may be conductive. For example, the non-force sensitive material may include a metal foil.

In some embodiments, the laryngoscopy device may include a voltage step-up circuit coupled to the body portion. The voltage step-up circuit may be configured to receive a first supply voltage from the laryngoscope battery interface as an input and to output a second, higher, supply voltage to the microcontroller.

The body portion of the laryngoscopy device may include a base portion and an enclosure portion. The base portion may have an interior side, configured to be coupled to the laryngoscope blade, and an exterior side, configured to be coupled to the enclosure portion. The circuit may be coupled to the exterior side of the base portion, positioning the circuit between the base portion and the enclosure portion. A cushion layer may be coupled to the interior side of the base portion so that the cushion layer is positioned between the interior side of the base portion and the laryngoscopy blade, when the laryngoscopy blade is coupled to the interior side of the base portion. In some embodiments, the base portion may include a spacer portion positioned adjacent to a lateral side of the laryngoscope when the base portion is coupled to the laryngoscope blade. The spacer portion may include a conduit to allow wiring to run from the force sensor to the circuit.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent. In the disclosed embodiment, the term “adjacent” is generally defined located in the same discrete chamber, housing, or module.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system or apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a structure (e.g., a component of an apparatus) that is configured in a certain way is configured in at least that way, but it can also be configured in other ways than those specifically described.

Any embodiment of any of the present systems, apparatuses, and methods can consist of or consist essentially of—rather than comprise/include/contain/have—any of the described steps, elements, and/or features. Thus, in any of the claims, the term “consisting of’ or “consisting essentially of’ can be substituted for any of the open-ended linking verbs recited above, in order to change the scope of a given claim from what it would otherwise be using the open-ended linking verb.

Details associated with the embodiments described above and others are presented below.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 is a perspective view illustrating a prior art laryngoscope.

FIG. 2 is a side view illustrating a prior art laryngoscope with onboard electronics and the blade folded at the blade attachment site.

FIGS. 3A-3D are perspective views of a force sensitive laryngoscope sensor coupled to a laryngoscope according to one embodiment of the disclosure.

FIGS. 4A-4E are perspective views of the body portion of a force sensitive laryngoscope sensor according to one embodiment of the disclosure.

FIGS. 5A-5C are perspective views of the base portion of a force sensitive laryngoscope sensor coupled to various laryngoscopes in open positions according to one embodiment of the disclosure.

FIGS. 6A-6C are perspective views of the base portion of a force sensitive laryngoscope sensor coupled to various laryngoscopes in closed positions according to one embodiment of the disclosure.

FIGS. 7A-7C are perspective views of the enclosure portion of a force sensitive laryngoscope sensor coupled to various laryngoscopes in closed positions according to one embodiment of the disclosure.

FIG. 8 is a schematic drawing of a voltage step up circuit, respectively, according to one embodiment of the disclosure.

FIGS. 9A and 9B are schematic drawings of a force sensing circuit, respectively, according to one embodiment of the disclosure.

FIG. 10 is a schematic drawing of a comparator according to one embodiment of the disclosure.

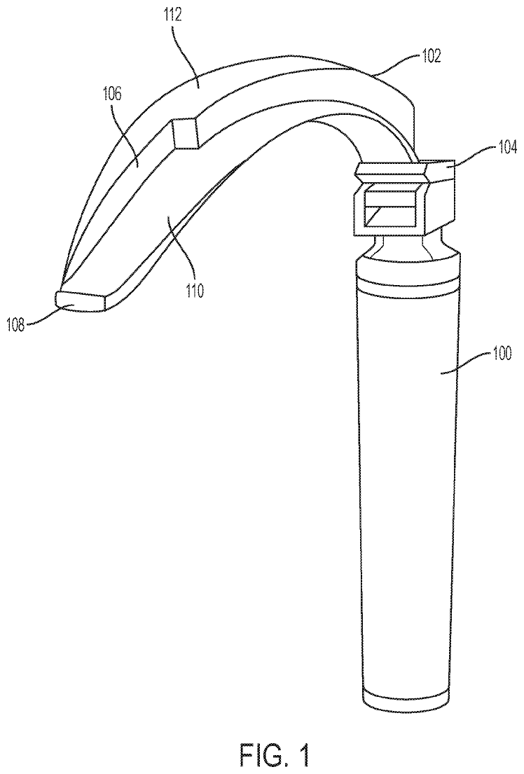

### DETAILED DESCRIPTION

Endotracheal intubation is one of the most common medical procedures, being performed on millions of people every year. The majority of tracheal intubations are performed in operating rooms by anesthetists or nurse anesthetists. One technique for tracheal intubation is direct laryngoscopy (DL). Indications for endotracheal intubations by DL include acute respiratory failure, inadequate oxygenation or ventilation, and airway protection in a patient with depressed mental status. As described above, laryngoscopes are commonly used to perform DL. During intubation procedures, laryngoscopes may, inadvertently, lead to dental damage in a large number of patients. This disclosure describes embodiments of methods, apparatuses, and systems for endotracheal intubation using a force sensitive laryngoscope sensor to better protect patients from complications, such as dental damage, during intubation procedures or other activities involving laryngoscopes.

An intubation device that reduces the risk of dental damage during intubation may provide benefits during direct laryngoscope (DL). In some embodiments, the intubation device is configured to removably couple with a standard laryngoscope and provide protection against dental damage. In some embodiments, the intubation device removably couples with the dorsal aspect and/or blade of a laryngoscope. In some embodiments, the intubation device is powered by the electrical system of the laryngoscope, such as by the laryngoscope's battery.

In some embodiments, the intubation device integrates a force sensor with a microcontroller to create a flexible, disposable, and accurate pressure sensitive add-on to conventional laryngoscopes. Such an add-on is designed to prevent dental damage through physical characteristics of the intubation device and/or warnings to the physician. In some embodiments, the microcontroller allows for accurate data readings and quick transfer to an external output. Additionally, the low voltage requirement of the microcontroller may allow for an onboard miniature low cost battery source. Alternatively, in some embodiments, the intubation device is configured to powered by the existing power source of the attached laryngoscope, such as a battery of the laryngoscope. In some embodiments, the device includes pressure sensing material comprising force sensitive polymeric material. Such pressure sensitive material may be configured to come into direct contact with an external force, such as the teeth of a patient when the device is being used to aid in DL.

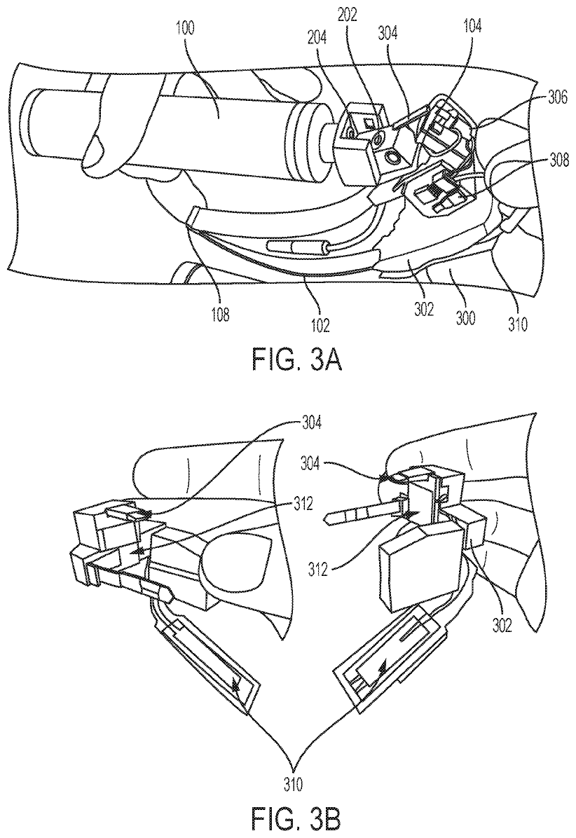

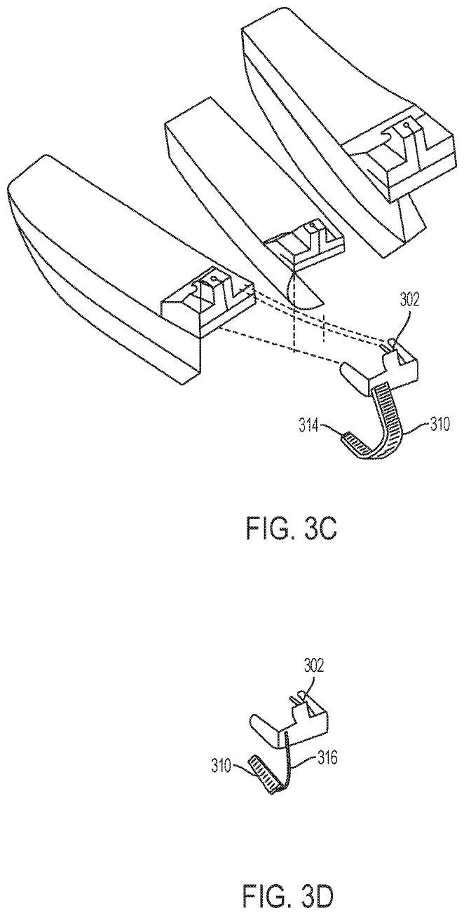

Referring now to the drawings, FIGS. 3A-3D show embodiments of the present intubation device. In the embodiments shown, device 300 comprises a body portion 302 configured such that the device 300 may be removably coupled to the blade portion 102 of a laryngoscope. In some embodiments, the device 300 is coupled to the blade portion 102 of the laryngoscope through a friction fit. The device 300 may be coupled to the laryngoscope by placing the body portion 302 onto the blade from the back end of the blade.

The body portion 302 may be formed to fit the shape of the dorsal aspect of the blade portion 102 of the laryngoscope. The body portion 302 extends from approximately the midpoint of the blade portion 102 of the laryngoscope such that the tip 108 is not covered, and extends proximally around the back of the laryngoscope ending at the blade attachment site 104. The device 300 may include a battery interface 304 located between the electrical contacts 202, 204 of the laryngoscope such that when electricity flows from the battery located in the handle 100 to the blade portion 102, the electricity flows into the device 300 via the battery interface 304. In the embodiment shown, the battery interface 304 is in electrical communication with a voltage step-up circuit 306, or other voltage regulation circuit that receives a first supply voltage and outputs a second supply voltage, which is in electrical communication with a force sensing and alarm buzzer circuit 308. The voltage step up circuit 306 shares a common ground 312 with the laryngoscope. The force sensing and alarm buzzer circuit 308 is in electrical communication with and receives input from a force sensor 310. In the embodiment shown, the force sensor 310 runs along the dorsal aspect of the blade portion 102 of the laryngoscope and is coupled to the body portion 302 of the device 300. In some embodiments, the force sensor 310 may cover a majority or all of the dorsal aspect of the blade portion 102 of the laryngoscope to prevent dental damage along its length. For example, the force sensor 310 may encase the majority of the blade portion 102 of the laryngoscope, including a lateral portion of the blade 102.

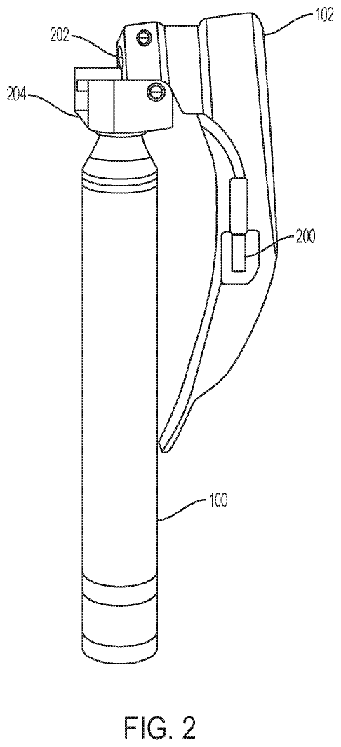

The device 300 is powered by a battery located in the handle 100 of a laryngoscope. The battery may be included in laryngoscopes to power features of the laryngoscope, including lights located on the blade portion 102 as shown in FIG. 2. The batteries may be disposable or rechargeable. The electrical components (including the force sensor 310 and accompanying force sensing and alarm buzzer circuit 308) may be automatically turned on when the device 300 is connected to a laryngoscope through the battery interface 304 between the electrical contacts 202, 204 to allow electricity to flow to the device. The device 300 may include an on/off switch (not shown) to selectively power on the device after the device 300 is placed on a laryngoscope. In some embodiments, the device 300 may include an on-board battery system separate from any battery system present in the attached laryngoscope. In this configuration, the device 300 may include a sensor that detects when the device is coupled with the laryngoscope to automatically power on the electronic components when attached. Alternatively, embodiments with an on-board battery system may additionally have an on/off switch to allow the user to selectively power on the device after the device is placed on a laryngoscope.

The battery interface 304 may have a conductive electrode extension from the body portion 302 that connects the laryngoscope's battery to the electronics of the disclosed device. The conductive electrode extension may be rigid or flexible. The battery interface 304 may be a single conductive layer. Alternatively, the battery interface 304 may be two-sided and configured to allow for normal electric flow through the laryngoscope from the handle 100 to the blade 102, rather than shunting the electricity just to the device. The conductive electrode extension may have a loop shape and/or be constructed of mesh to avoid blocking the light in fiber optic laryngoscope models. Alternatively, the conductive electrode extension may be formed in another shape that will allow enough light to pass through to avoid impeding the operation of a flashlight portion of the blade 102. Portions of the conductive electrode that touch parts of the laryngoscope blade 102 that are not a part of the battery may be covered by an insulation layer to prevent contact with the device's electric ground.

The body portion 302 may be configured to be an inverse mold made of latex, rubber, or another flexible material of the back end of a laryngoscope blade 102. The body portion 302 may be configured to provide for a close fit onto the blade 102, while simultaneously facilitating easy removal by users. A close fit may be a fit having no slippage between the device 300 and the blade 102. In some embodiments, the latex is liquid latex rubber, but may be another disposable, non-toxic, non-inflammatory, flexible, stretchable, sterilizable material thick enough to provide protection against dental trauma. The body portion 302 serves as a platform for the electronics (including the battery interface 304, the voltage step-up circuit 306, the force sensing and alarm buzzer circuit 308, and/or the force sensor 310), but may also have dental trauma prevention characteristics by itself. For example, the latex mold of the body portion 302 may be formed by adding filler material (e.g., strips of gauze) to the mold during a layer by layer curing process. Such filler material increases the overall durability of the body 302, thereby increasing the overall life-span of the device 300. In some embodiments, each layer of the mold has a thickness of less than 1, 2, 3, 4, or 5 millimeters and the mold includes ten layers applied across a 24-hour period to create the mold. Other manufacturing techniques may be used, such as manufacturing the body portion 302 by 3D printing using nylon or acrylonitrile butadiene styrene (ABS). Some other manufacturing techniques may form the body 302 from silicone or polydimethylsiloxane (PDMS). In some embodiments, the body portion 302, when coupled to the laryngoscope, covers the posterior portion of the blade 102, such as to guard or protect a portion or the entirety of a posterior portion of the blade 102.

FIGS. 3C and 3D depict embodiments of the body portion 302 of the device including a force sensor 310. According to some embodiments, as shown in FIG. 3C, the device comprises a force sensitive tape 314 configured to be used as the force sensor 310. According to some embodiments, as shown in FIG. 3D, the device comprises a wire 316 configured to electrically couple the electronics of the body portion 302 with the force sensor 310.

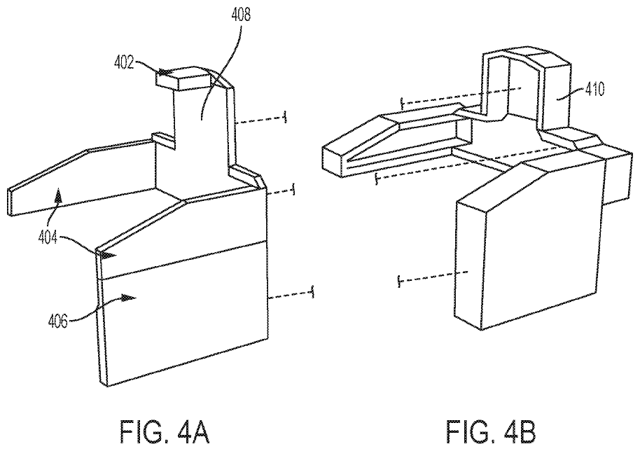

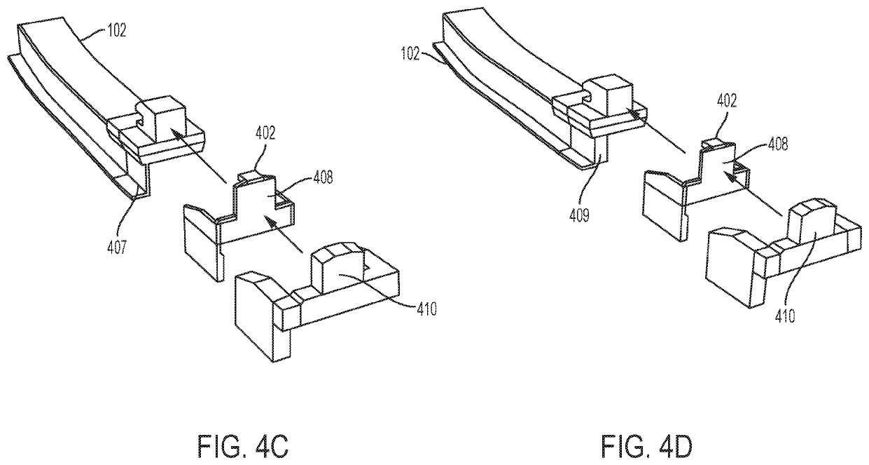

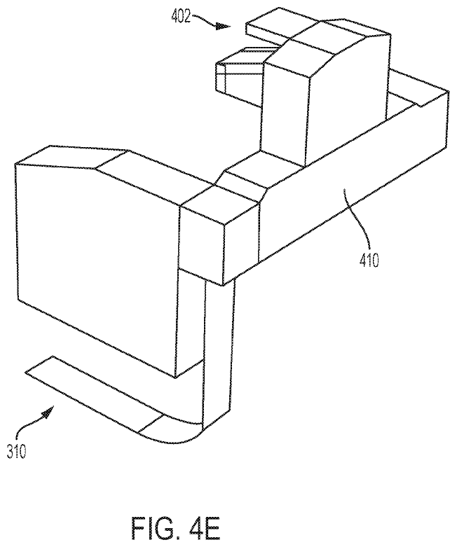

FIGS. 4A and 4B depict an embodiment of the body portion of the device. The body portion 302 may have a base 408 and an enclosure 410. In some embodiments, the base interfaces with the proximal end of the laryngoscope blade between the horizontal flange 112 and the blade attachment site 104. The generalizable clip 408 has two arms 404 that wrap around the sides of the laryngoscope thereby connecting the handle hook to the rest of the blade. The electronics may be placed anywhere on the base 408. The base 408 may additionally include an extended platform 406 configured to hold more electronics or configured to assist with slipping the device onto the laryngoscope. The enclosure 410 may include a cushion backing and be configured to snap onto the base 408 by using a superior latch 402. For example, the superior latch 402 may be less than 1 mm thick. A thin superior latch 402 with a thickness of less than 1 mm may reduce the likelihood of disengagement of the latch due to applied pressure from an external source, such as a mouth of a patient. The cushion backing may be formed from rubber or any compressible material. The enclosure 410 when coupled to the base 408 is configured to cover some or all of the electronics coupled to the base. When all electronics are covered, the enclosure 410 leaves only the force sensor 310 and battery interface 304 exposed. The base 408 and enclosure 410 are configured to be secured to regions of the laryngoscope to prevent dislodging into the airway or interference with the user's view of the airway.

FIGS. 4C-4E depict embodiments of the body portion of the device and an interface between the body portion and the blade portion 102 of a laryngoscope. In some embodiments, a base 408 is configured to be removably coupled with a blade 102 of a laryngoscope and further configured to be coupled with an enclosure 410. FIG. 4C depicts an exploded view of a base 408 and enclosure 410 configured to be coupled to the blade 102 of a thin Ondontomed laryngoscope with width 407. FIG. 4D depicts an exploded view of a base 408 and enclosure 410 configured to be coupled to the blade 102 of a thick Heine laryngoscope with width 409. FIG. 4E depicts a perspective view of an embodiment of the body portion of the device where a base is coupled with an enclosure. In some embodiments, the latch 402 of the base and a tape-based force sensor 310 are visible while the enclosure 410 is coupled to the base.

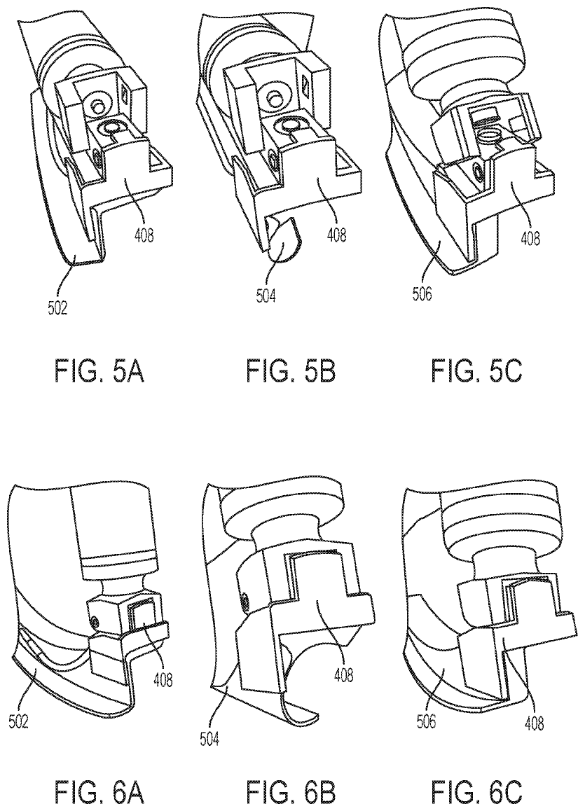

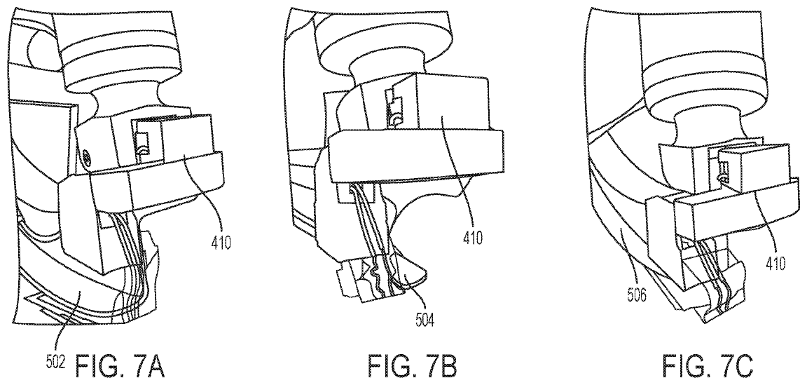

The body portion 302 may be configured to fit a wide variety of laryngoscope blade structures, not just a single laryngoscope model. In some embodiments, an expanded flexible mold may comprise flexible cushion interiors configured to be compressed to allow for a secure friction fit for a range of widths along all portions of the blade. For example, multiple layers of silicon cushion may be included to allow for such a secure friction fit. In some embodiments, the body portion 302 may be configured to fit a specific model of laryngoscope. FIGS. 5A-5C depict embodiments of the base 408 coupled to laryngoscope blades in an open position. FIG. 5A depicts the base 408 coupled to an Ondontomed Macintosh laryngoscope blade 502. FIG. 5B depicts the base 408 coupled to an Ondontomed Miller laryngoscope blade 504. FIG. 5C depicts the base 408 coupled to an Heine Macintosh 3 laryngoscope blade 506. FIGS. 6A-6C depict embodiments of the base 408 coupled to laryngoscope blades (Ondontomed Macintosh 502 in FIG. 6A, Ondontomed Miller 504 in FIG. 6B, Heine Macintosh 3 506 in FIG. 6C) in an closed position. FIGS. 7A-7C depict embodiments of the enclosure 410 coupled to laryngoscope blades (Ondontomed Macintosh 502 in FIG. 7A, Ondontomed Miller 504 in FIG. 7B, Heine Macintosh 3 506 in FIG. 7C) in a closed position. In some embodiments, the base 408 may be configured to attach to a video laryngoscope and to prevent interference with a view of the video laryngoscope.

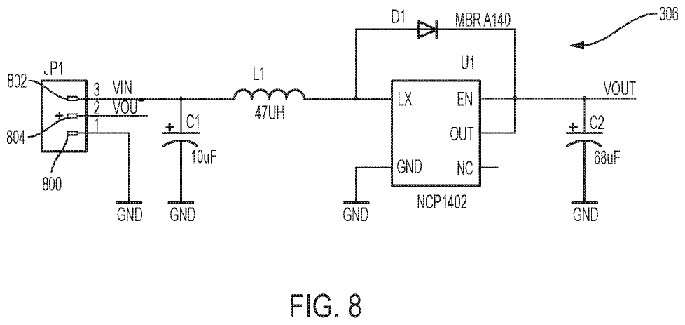

One electronics configuration for the device 300 may include two stages of operation: a boosting stage and a microcontroller stage. The boosting stage includes a voltage step-up circuit, such as a 3.3V or 5V step-up circuit. The step-up circuit serves to boost the voltage of the laryngoscope's batteries to a suitable voltage for microcontroller operation. FIG. 8 depicts an embodiment of the voltage step-up circuit 306. The voltage step-up circuit 306 includes three interfacing pins: ground pin 800, voltage in pin 802, and voltage out pin 804. The ground connection 800 is in contact with the laryngoscope blade 102 via a connector which allows for the laryngoscope and step-up circuit 306 to share a common ground 312, a requirement for interfacing with the laryngoscope's built-in battery system. The voltage in pin 802 is connected to the laryngoscope's battery contact 202 that is exposed at the blade attachment site 104 via the battery interface 104. The voltage out pin 804 delivers the stepped-up voltage from the laryngoscope's batteries and powers the force sensing and alarm buzzer circuit 308. In one embodiment, the device 300 is powered by batteries coupled to the body 302 which are separate from the laryngoscope's batteries. In some embodiments, the device 300 may be selectively powered by batteries coupled to the body 302 which are separate from the laryngoscope's battery or the laryngoscope's battery. For example, the device 300 may include a switch (not shown) for selecting between powering the device with an battery of the device 300 or a battery of the laryngoscope. Alternatively or additionally, the device 300 may be configured to use the battery of the laryngoscope as a backup battery, when the charge of a battery of the device 300 is depleted or the battery is not present.

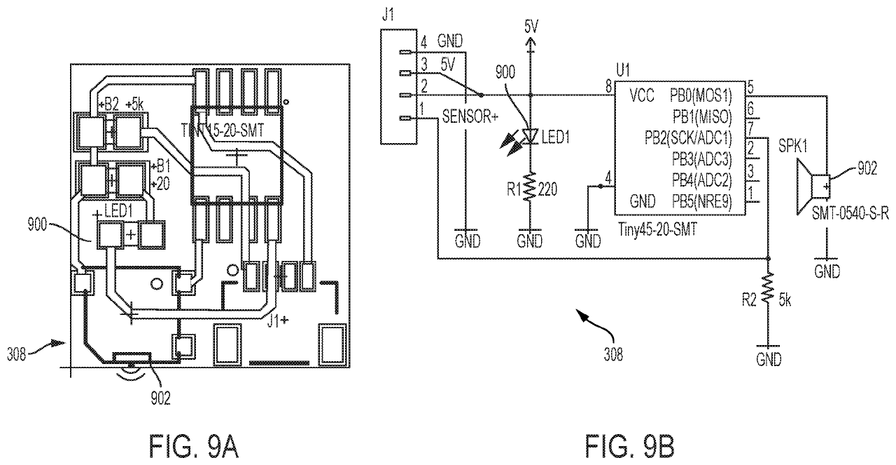

The microcontroller stage of the disclosed device's electronics may be powered from the voltage out pin 804 and include a force sensing circuit powered by the output from the voltage step-up circuit of the boosting stage. FIG. 9A depicts an embodiment of the force sensing circuit 308, and FIG. 9B depicts a circuit diagram of one embodiment of the force sensing circuit 308. The force sensing circuit 308 comprises a parallel “power on” LED 900 indicating when the circuit 308 is powered on and ready to use. For example, the LED 900 may turn on as a result of the device 300 being positioned on the blade 102 of a laryngoscope and the blade 102 being set in the upright position for intubation. When so positioned and configured, battery interface 304 is positioned between the electrical contacts 202, 204 thereby powering the voltage step-up circuit 306 and the force sensing circuit 308. In some embodiments, the force sensing circuit 308 is configured to activate a speaker 902 when a force is sensed by the force sensor 310. The speaker 902 may output various noises proportional to an amount of applied force, such as a soft intermittent buzzing as a safe threshold is reached and a loud constant buzzing when the threshold is exceeded. In some embodiments, the overall current consumption of the device 300 is approximately 50 mA when the force sensing circuit 308 is powered on, 280 mA when the speaker 902 is activated, and 0 mA when the force sensing circuit 308 is powered off. When the device 300 is implemented into standard laryngoscope battery systems, the device may have up to or above approximately 10 hours of usage before the batteries need to be replaced. Such a battery life allows for a very large number of intubation procedures to be performed, each of which take at most a few minutes to perform.

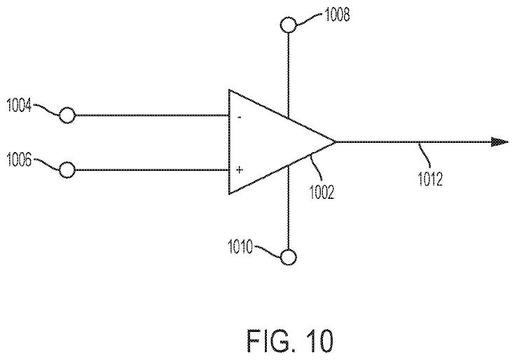

Another electronics configuration for the device 300 may include a comparator 1002 that receives input from a force sensor 310 and activates a buzzer 902 when force is detected. FIG. 10 depicts a circuit diagram of one embodiment of the comparator 1002. The comparator 1002 comprises a sensor voltage input 1004, a reference voltage input 1006, a positive supply rail 1008, a negative supply rail 1010, and an output 1012. In some embodiments, the comparator 1002 is electrically connected via the sensor voltage input 1004 to a force sensor 310. The input received from the force sensor 310 is compared to a reference voltage received by the comparator 1002 at the reference voltage input 1006. When the input from the force sensor 302 reaches a predetermined level, the comparator 1002 outputs a signal to a buzzer or speaker 902 that is electrically connected to the output 1012. The positive supply rail 1008 is electrically connected to a power supply, which may include a battery system coupled to the body portion 302 of the disclosed apparatus or the battery of a laryngoscope via the battery interface 304. The negative supply rail 1010 is connected to a ground.

In another electronics configuration, the comparator 1002 may additionally be electrically connected to a voltage step-up circuit 306 to boost or maintain the initial operating voltage at either the reference voltage input 1006 and/or the positive supply rail 1008. In some embodiments, a potentiometer or trimmer resistor may be used for device calibration. Such a potentiometer or trimmer resistor may be used to tune the resistor used to reference the voltage across the force sensor 302.

In some embodiments, the force sensor 310 is formed by layering with alternating layers of a force sensitive conductive material and a conductive non-force sensitive material. In some embodiments, the force sensitive conductive material is Velostat sheets and/or the conductive non-force sensitive material is a metal foil (e.g., aluminum foil). The outermost layers of the force sensor are the force sensitive conductive material, where two interfacing wires are taped down (or otherwise electrically coupled), one on each side of the overall sensor. These contacts correspond to the positive and negative contacts of the force sensor and are coupled to the force sensing circuit 308. In some embodiments, the positive contact is coupled to the 5-volt source at the voltage out pin 804 and the negative contact is placed in series with another resistor. The pressure may be sensed though a voltage divider configuration. The force sensor's resistance value changes from a high mega-ohms range (e.g., millions of ohms) to a low kilo-ohms range (e.g., thousands of ohms) upon contact with external pressure. The threshold for activating the speaker may be set by the series resistance of 5000 ohms and by providing the voltage divider with 5 volts. When the force sensitive resistance drops to a value lower than 5000 ohms, the voltage across the 5000-ohm resistor becomes greater than 2.5 volts. The voltage may be read by the force sensing circuit 308 and if it is below a set threshold (e.g., 2.5 volts) the speaker is activated to alarm the user of the dangerous pressure being applied. The threshold level and resistance level may be configured for different applications and different patients. In some embodiments, multiple threshold levels may be configurable such that a user is provide a green/yellow/red status or a numerical value regarding the applied force.

In some embodiments, the force sensor 310 comprises an extended platform 406, a force sensitive region, a securing structure, and a protective structure. The securing structure may comprise an adhesive, a clip with a friction fit running down the length of the force sensor 310, etc. that allows for securing the force sensor 310 to the desired location, while still allowing the force sensor to be removed and secured again without the ability to secure diminishing. The securing material may be magnetic or chemical based, or come in the form of a tape. The protective structure may comprise a cushioning material such as liquid latex surrounding the force sensitive region, or any nearby region that potentially interfaces with the teeth of a patient.

As shown in FIGS. 4A and 4B, the extended platform 406 may act as a spacer to allow for the force sensor 310 to be positioned over the necessary region. The extended platform 406 may be plastically laminated to a thickness small enough to be flexible and allow for bending around structures placed on the target region. Alternatively, the spacer region may comprise a metal tubing containing the wires connecting the force sensor 310 to the other electronics, where the tubing may be flexible enough for manual manipulation to fit the force sensor 310 on the desired location, while still rigid enough to maintain that position once so configured. According to some embodiments, the tubing may be constructed from aluminum or any other bendable metal or plastic.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. An apparatus, comprising:
a body portion having an interior side and an exterior side;
a force sensor coupled to the exterior side of the body portion;
a circuit coupled to the force sensor and configured to provide feedback based on an output of the force sensor; and
a laryngoscope battery interface,
wherein the body portion is configured to be removably connected to a laryngoscope having a laryngoscope blade such that the interior side of the body portion is adjacent to the laryngoscope blade, and
wherein the laryngoscope battery interface is configured to electrically couple the circuit to a power system of a laryngoscope when the body portion is attached to the laryngoscope.

2. The apparatus of claim 1, wherein the apparatus is disposable.

3. The apparatus of claim 1, wherein the body portion comprises latex rubber.

4. The apparatus of claim 1, wherein the force sensor comprises a plurality of layers of force sensitive material.

5. The apparatus of claim 4, wherein the force sensitive material is conductive.

6. The apparatus of claim 4, wherein the force sensor further comprises a non-force sensitive material.

7. The apparatus of claim 6, wherein the non-force sensitive material is conductive.

8. The apparatus of claim 7, wherein the non-force sensitive material is a metal foil.

9. The apparatus of claim 1, wherein the circuit comprises a microcontroller configured to detect when pressure applied to the force sensor exceeds a first threshold level.

10. The apparatus of claim 9, further comprising a voltage step-up circuit coupled to the body portion and configured to receive a first supply voltage from the laryngoscope battery interface as input and output a second, higher, supply voltage to the microcontroller.

11. The apparatus of claim 9, wherein the microcontroller is configured to operate a feedback device to emit a first tone when the pressure applied to the force sensor exceeds the first threshold level and a second tone when the pressure applied to the force sensor exceeds a second threshold level.

12. The apparatus of claim 1, wherein the circuit comprises a comparator configured to detect when pressure applied to the force sensor exceeds a first threshold level.

13. The apparatus of claim 1, wherein:
the body portion further comprises a base portion and an enclosure portion;
the base portion comprises an interior side and an exterior side;
the exterior side of the base portion is configured to be coupled to the enclosure portion; and
the interior side of the base portion is configured to be coupled to the laryngoscope blade.

14. The apparatus of claim 13, wherein the circuit is further coupled to the exterior side of the base portion such that the circuit is positioned between the base portion and the enclosure portion when so coupled.

15. The apparatus of claim 13, further comprising a cushion layer coupled to the interior side of the base portion such that the cushion layer is positioned between the interior side of the base portion and the laryngoscope blade when so coupled.

16. The apparatus of claim 1, wherein the force sensor is configured to be coupled to the laryngoscope blade via a securing structure.

17. The apparatus of claim 16, wherein the securing structure comprises a clip that couples the force sensor to the laryngoscope blade via friction.

18. The apparatus of claim 16, wherein the securing structure comprises an adhesive.

19. The apparatus of claim 13, wherein the base portion further comprises a spacer portion adjacent to a lateral side of the laryngoscope when the base portion is coupled to the laryngoscope blade.

20. The apparatus of claim 19, wherein the spacer portion comprises a conduit configured to allow wiring to run from the force sensor to the circuit.
